(19) United States
(12) Patent Application Publication
(10) Pub. No.: US 2004/0086564 A1
(43) Pub. Date: May 6, 2004

(54) Dosage forms containing stabilized choline and method for preparing same

(76) Inventors: Paul H. Richardson, McAfee, NJ (US); Lucien Hernandez, Bronx, NY (US)

Correspondence Address: HOFFMANN & BARON, LLP, 6900 JERICHO TURNPIKE, SYOSSET, NY, 11791, US

(21) Appl. No.: 10/285,053

(22) Filed: Oct. 31, 2002

(51) Int. Cl. A61K 9/00; A61K 9/26; B29C 39/10

(52) U.S. Cl. 424/469; 264/4

(57) ABSTRACT

The present invention provides a dosage form containing stabilized choline and a method for preparing the dosage form. The choline is stabilized by encapsulating a low hygroscopic choline salt in a lipid coating.

### BACKGROUND OF THE INVENTION

The present invention relates to the art of providing nutritional ingredients to enhance well-being, and, in particular, to the preparation and delivery of choline as a nutritional product.

Choline is an essential nutrient frequently referred to as vitamin B4. Choline is important for the structural integrity of cell membranes, methyl metabolism, cholinergic neurotransmission, transmembrane signaling, lipid-cholesterol transport and metabolism. It also enhances memory, promotes heart function by reducing plasma homocysteine levels (a known cardiovascular risk factor), and promotes liver function by preventing fatty deposits. A deficiency in choline can lead to hypertension, arteriosclerosis, cirrhosis and fatty degeneration of the liver. Therefore, due to the many health benefits provided by choline, it is rapidly becoming a desired part of any health-conscious diet.

Choline can be found in a number of foods. Egg yolk, meat, fish, cereals, and legumes all contain at least some trace of the nutrient. The richest sources of choline, however, are typically foods high in fat and cholesterol. Thus, the health benefits of choline may be outweighed by other health concerns associated with a high fat and cholesterol diet. Furthermore, adopting a low-fat diet may reduce one's choline intake to suboptimal levels.

Dosage forms, such as tablets and capsules, containing choline can be used to supplement one's diet. Several patents disclose tablets containing choline. U.S. Pat. No. 6,110,501 to Redding, Jr. et al., discloses a tablet containing lipid encapsulated choline, specifically choline chloride. Seeds are dispersed throughout the shell of the encapsulate to increase the strength of the encapsulate during manufacturing of a tablet.

U.S. Pat. No. 4,626,527 to Wurtman et al. discloses administering choline to a patient. The choline can be administered in the form of a tablet or capsule.

U.S. Pat. No. 6,361,800 B1 to Cooper et al. discloses a multi-vitamin and mineral supplement containing choline and heavy metals, such as copper, selenium, zinc, and chrominum. The choline is preferably choline bitartrate. The nutritional components (e.g., vitamins, minerals) used in the supplement are blended with an excipient, such as vegetable oil.

Studies to date, however, have shown that multi-vitamin and mineral tablets containing unencapsulated choline salts, encapsulated hygroscopic choline salts such as choline chloride, as well as poorly encapsulated non-hygroscopic choline salts, are not stable during storage. It is believed that the interaction of choline with heavy metals present in the multi-vitamin and mineral tablet generally destabilizes choline. The loss of stability of choline results in decreased choline activity, and/or odor development and/or discoloration of the tablet during storage. This reduces the shelf-life of the product and is undesirable to the consumer.

Therefore, there is a need for a dosage form containing choline having a desired degree of protection. The level of protection stabilizes the choline against conditions encountered during manufacturing and storage of the dosage form.

### SUMMARY OF THE INVENTION

The present invention provides a dosage form containing stabilized choline and a method for preparing the dosage form. The choline, present in the form of a choline salt, is stabilized by encapsulation, and thus protects choline against conditions encountered during manufacturing and storage of the dosage form. The choline salt can be mechanically processed, such as roller compacted, granulated, or chilsonated, prior to encapsulation.

The choline salt can be any low hygroscopic choline salt, such as choline bitartrate, choline dihydrogen citrate, and combinations thereof. The choline salt can be USP (U.S. pharmacopeia) grade.

The choline salt is encapsulated with a lipid coating. The lipid can be any lipid, lipid derived material, waxes, organic esters, fatty alcohols, or combinations thereof. In a preferred embodiment, the lipid includes a hydrogenated vegetable oil, such as palm oil.

The lipid coating can further contain additives. The additive can be any additive which enhances the integrity of the coating, such as increased stability of the coating; or which adds to the functionality of the coating, such as the controlled release of choline (e.g., release due to pH, etc.) and enhanced resistance of the coating to compression. Preferably, the additives improve the tolerance of the encapsulate to pressures encountered during manufacture of the dosage form.

The lipid coating can also contain additives which enhance the organoleptic properties of the encapsulate. Examples of such additives include, but are not limited to, preservatives, flavors, and anti-oxidants.

The encapsulated choline has a 5 hour release of less than about 20% when exposed to water, preferable less than about 15%, more preferably less than about 10%, even more preferably less than about 5%. In a preferred embodiment, the 5 hour release is less than about 1%.

The encapsulated choline is combined with other ingredients to prepare a dosage form. The dosage form can be a tablet or gel capsule. In a preferred embodiment, the dosage form contains at least one heavy metal selected from the group consisting of copper, iron, magnesium, manganese, molybdenum, zinc, nickel, selenium, chromium, tin, vanadium, and combinations thereof.

In another preferred embodiment, the tablet or gel capsule is a multi-vitamin and mineral supplement.

The amount of encapsulated choline in a dosage form can be any amount which provides at least a health benefit. Preferably, the dosage form contains at least about 5 mg of choline cation, more preferably at least about 20 mg, even more preferably at least about 55 mg, and most preferably at least about 110 mg of choline cation.

As a result of the present invention, a dosage form is provided which contains stabilized choline. The choline is protected against conditions associated with manufacturing and storage of a dosage form. Therefore, the stabilized choline does not result in significant discoloration and odor development of the dosage form, nor any siginificant loss in nutrient activity.

For a better understanding of the present invention, together with other and further advantages, reference is made to the following detailed description, and its scope will be pointed out in the claims.

### DETAILED DESCRIPTION OF THE INVENTION

The present invention includes a dosage form containing stabilized choline and a method for preparing the dosage form. The choline, present in the form of a choline salt, is stabilized by encapsulation, which offers the choline a degree of protection against conditions encountered during manufacturing and storage of the dosage form.

Choline can exist in various salt forms, such as choline bitartrate and choline dihydrogen citrate. “Choline cation” as used herein refers to the choline portion of a choline salt. For example, choline bitartrate contains about 40% choline cations and choline dihydrogen citrate contains about 35% choline cations.

Choline salts useful in the present invention are those salts which exhibit how hygroscopicity. Low hygroscopic choline salts do not readily absorb moisture or water from the surrounding environment.

Low hygroscopic choline salts useful in the present invention include those salts which gain less than 10% by weight after exposure to 66% relative humidity, at room temperature for 24 hours, preferably gain less than about 5% by weight, and more preferably less than 2% by weight. Examples of choline salts with low hygroscopicity include, but are not limited to, choline bitartrate, choline dihydrogen citrate, and combinations thereof. The choline salt can be USP (U.S. pharmacopeia) grade.

The low hygroscopic choline salt can be mechanically processed prior to encapsulation. Examples of methods to mechanically process choline salts of the present invention include roller compaction, granulation, and chilsonation. These methods are known to those skilled in the art. Process aids, such as additives, can be employed during mechanical processing. An example of an additive is silicon dioxide. For example, silicon dioxide can be used in the granulation of a low hygroscopic choline salt.

A low hygroscopic choline salt is stabilized by encapsulating the choline salt with a lipid coating. The term “lipid” as used herein includes any lipid, lipid derived material, waxes, organic esters, fatty alcohols, or combinations thereof. The lipid can be derived from animals, vegetables, mineral, or synthetic origins. The lipid is preferably hydrogenated, and can be saturated or partially saturated, and includes, but is not limited to, mono-, di-, and triglycerides.

The wax can be paraffin wax; a petroleum wax; a mineral wax such as ozokerite, ceresin, utah wax or montan wax; a vegetable wax such as, for example, carnuba wax, japan wax, bayberry wax or flax wax; an animal wax such as, for example, spermaceti; or an insect wax such as beeswax, Chinese wax or shellac wax.

Additionally, the wax material can be an ester of a fatty acid having 12 to 31 carbon atoms and a fatty alcohol having 12 to 31 carbon atoms, the ester having from a carbon atom content of from 24 to 62, or a mixture thereof. Examples include myricyl palmitate, cetyl palmitate, myricyl cerotate, cetyl myristate, ceryl palmitate, ceryl certate, myricyl melissate, stearyl palmitate, stearyl myristate, and lauryl laurate.

The fatty acids can have from 10 to 22 carbon atoms and can be, for example, decenoic, docosanoic, stearic, palmitic, lauric or myristic acid.

The fatty alcohols can have from 14 to 31 carbon atoms and can be, for example, lauryl, cetyl, stearyl, myristyl, myricyl, arachyl, carnubyl or ceryl alcohol.

The fatty acid esters can be mono-, di-, or triglyceryl esters formed from fatty acids having from 10 to 22 carbon atoms, such as for example glyceryl distearate, glyceryl tristearate, glyceryl monostearate, glyceryl dipalmitate, glyceryl tripalmitate, glyceryl monopalmitate, glyceryl dilaurate, glyceryl didocosanoate, glyceryl tridocosanoate, glyceryl monodocosanoate, glyceryl monocaprate, glyceryl dicaprate, glyceryl tricaprate, glyceryl monomyristate, glyceryl dimyristate, glyceryl trimyristate, glyceryl monodecenoate, glyceryl didecenoate, or glyceryl tridecenoate.

Preferred coatings comprise hydrogenated vegetable oils including triglycerides such as hydrogenated cottonseed, corn, peanut, soybean, palm, palm kernel, babassu, sunflower, and safflower oils. Preferred hydrogenated vegetable oils include hydrogenated palm oil, cottonseed oil, and soybean oil. Other vegetable-, animal-, mineral-, and synthetic-derived fats and waxes also are suitable.

The lipid coating can be a mixture of different lipids. For example, in addition to the preferred hydrogenated vegetable oils, other lipids which can be incorporated in the lipid coating include beeswax, petroleum wax, and lower melting hydrogenated vegetable oil blends. Other waxes and oils such as rice bran wax and castor wax also are suitable components in the lipid coating of the present invention.

The lipid coating can further contain additives. The additive can be any additive which enhances the integrity of the coating, such as increased stability of the coating; or which adds to the functionality of the coating, such as the controlled release of choline (e.g., release due to pH, etc.), and enhanced resistance of the coating to compression. Preferably, the additives-improve the tolerance of the encapsulate to pressures encountered during manufacture of the dosage form. Examples of additives useful in the present invention include, but are not limited to, mono- and diglycerides.

Furthermore, the lipid coating can also contain additives which enhance the organoleptic properties of the encapsulate. For example, the additives can enhance the taste, color, and texture of the coating. Examples of such additives include, but are not limited to, preservatives, flavors, and anti-oxidants.

The method of applying the coating to the choline salt is not critical, forms no part of the present invention, and can be performed in any manner so long as the coating provides the choline salt with the desired degree of protection and release, such that the choline is stabilized during manufacture and storage of the dosage form. The low hygroscopic choline salt can be encapsulated in a lipid by any method known to those in the art. For example, choline salt granules can be suspended in the molten lipid and the suspension sprayed into a “freezing chamber.”

Alternatively, the choline salt can be coated with a molten lipid in a fluidized bed apparatus. U.S. Pat. No. 4,511,584 at columns 3-5, U.S. Pat. No. 4,537,784 at columns 4-5; U.S. Pat. No. 4,511,592 at column 4, and U.S. Pat. No. 4,497,845 at column 4, disclose methods of applying a lipid coating to granular particles in a fluidized bed apparatus. In essence, granular particles are introduced into a fluidized bed chamber. The coating material is then applied to the granular particles by spraying the coating material into the fluidized bed chamber. The methods disclosed in U.S. Pat. No. 4,511,584, U.S. Pat. No. 4,537,784, U.S. Pat. No. 4,511,592, and U.S. Pat. No. 4,497,845 can be adapted for applying a lipid coating to choline salts in a fluidized bed apparatus. The relevant portions of U.S. Pat. No. 4,511,584, U.S. Pat. No. 4,537,784, U.S. Pat. No. 4,511,592, and U.S. Pat. No. 4,497,845 are hereby incorporated by reference.

The level of protection of choline depends on the nature (e.g., type and amount of lipid, process of encapsulation, etc.) of the encapsulate coating. The level of protection of the choline, which is useful in the present invention, are those encapsulated cholines which have a choline release of less than about 20% when the encapsulate is exposed to water for five hours, preferably less than about 15%, more preferably less than about 10%, even more preferably less than about 5%. In a preferred embodiment, the release is less than about 1% when exposed to water for five hours.

The amount of choline salt (e.g., activity of choline) present in an encapsulate can be any amount such that the release of choline from the encapsulate is in accordance with the present invention, as discussed above. Preferably, the choline is present in the encapsulate at a minimum amount of about 30% by weight of the encapsulate, preferably about 50% by weight, and more preferably about 70% by weight of the encapsulate.

The encapsulated cholines having a release in accordance with the present invention are thus stabilized against conditions encountered during manufacturing and storage of a dosage form. Conditions encountered during manufacturing and storage of a dosage form can include, for example, exposure to moisture, oxygen, elevated temperature, elevated pressure, and interaction with other ingredients present in the dosage form.

Due to stabilization of choline, little to no odor development or discoloration of the dosage form occurs during storage. As a result, the shelf-life of the dosage form is about 2 years when exposed to conditions typically encountered during storage, distribution, and use.

In a preferred embodiment, the choline salt is stabilized for at least about three months when the dosage form is stored under accelerated storage conditions. The term “accelerated conditions” as used herein means a temperature of approximately 40° C. and a relative humidity of approximately 75%.

To prepare a dosage form of the present invention, a plurality of stabilized encapsulated choline are combined with other ingredients. Dosage forms include, for example, tablets and gel capsules. Tablets include, for example, pills, caplets, and capsule shaped tablets.

The amount of encapsulated choline in a dosage form can be any amount which provides at least a health benefit. Examples of health benefits from choline include reducing plasma homocysteine levels, promoting liver function, enhancing memory. Preferably, the dosage form contains at least about 5 mg of choline cation, more preferably at least about 20 mg, even more preferably at least about 55 mg, and most preferably at least about 110 mg of choline cation.

The other ingredients useful for preparing a dosage form are known to those in the art. For example, the dosage form can contain excipients, such as plasticizers. Examples of plasticizers include diethylphthalate, dibutyl sebacate, triethyl citrate, triacetin, vegetable oils, polyethylene glycol, and combinations thereof. Preferably, the plasticizer, when present in the dosage form, is present at a minimum amount from about 0.01%, more preferably about 0.1%, and most preferably from about 1% by weight of the dosage form. The plasticizer is present at a maximum amount from about 25%, more preferably about 10%, and most preferably from about 5% by weight of the dosage form.

The other ingredients can also include fillers. Fillers can, for example, be used to increase the bulk of a tablet to render the combination of ingredients suitable for compression. Examples of fillers include calcium sulfate, calcium carbonate, dicalcium phosphate, modified starches, maltodextrin, sucrose, lactose, manitol, sorbitol, microcrystalline cellulose, and combinations thereof. The fillers are present in the dosage form at a minimum amount of about 2%, more preferably about 10%, and most preferably about 20% by weight of the tablet or gel capsule. The fillers are present in the dosage form at a maximum amount of about 70%, more preferably about 50%, and most preferably about 40% by weight of the dosage form.

Other ingredients can also include binders. Typically, binders contribute to the ease of comprssion and general quality of, for example, a tablet. Examples of binders include starches, pregelatinize starches, gelatin, polyvinylpyrrolidone, methylcellulose, sodium carboxymethylcellulose, ethylcellulose, polyacrylamines, polyvinyloxoazolidone, and polyvinylalcohols.

Other ingredients, such as coloring agents, preservative, sweeteners both artificial and natural, and flavorants can also be used to prepare the dosage form. The amounts and combinations of the ingredients useful for preparing a dosage form are known to those skilled in the art.

If the dosage form (e.g., tablet, gel capsule, etc.) is a multi-vitamin and mineral supplement, the other ingredients further include vitamins and minerals. The amounts and combinations of vitamins and minerals to include are known to those in the art. For example, the amount can be less than the U.S. recommended daily allowance (USRDA), more than the USRDA, or about the USRDA for a particular vitamin or mineral.

The vitamin can be any vitamin. Examples of vitamins include, but are not limited to, vitamin A (retinol), vitamin BI (thiamine), vitamin B2 (riboflavin), B complex vitamin, vitamin B6 (pyridoxine), vitamin B12 (cobalamin), vitamin C (ascorbic acid), vitamin D (cholecalciferol), vitamin E (tocopherol), vitamin F (linoleic acid), vitamin K, beta-carotene, biotin, folic acid, niacin, and pantothenic acid.

The mineral can be any mineral, preferably present in a salt form. Examples of minerals include, but are not limited to, boron, calcium, chromium, copper, iron, magnesium, manganese, molybdenum, nickel, phosphorus, selenium, silicon, tin, vanadium, and zinc.

In a preferred embodiment, the dosage form contains at least one heavy metal selected from the group consisting of copper, iron, magnesium, manganese, molybdenum, zinc, nickel, selenium, chromium, tin, vanadium, and combinations thereof.

If the dosage form is a tablet, the tablet can be prepared by any method known to those skilled in the art. For example, a mixture containing the stabilized encapsulated choline salt and other ingredients is compressed into a tablet form using a tabletting machine typically utilized in the pharmaceutical arts. The tabletting machine typically includes a die and a punch. The mixture is fed to the die cavity of a tablet press and sufficient pressure is applied by the punches to form a solid tablet. The pressure that is applied can vary. Generally, the pressure ranges from about 1,000 psi to about 6,000 psi. Preferably, the pressure is about 3,000 psi.

After the tablet is formed, the tablet can be coated with materials typically used in the pharmaceutical arts. The coating materials and the coating technique are known to those skilled in the art.

If preparing a gel capsule is desired, the manufacture of gelatin capsules are well known in the pharmaceutical arts. For example, a gelatin shell is filled with a mixture containing stabilized encapsulated choline salts and other ingredients.

### EXAMPLES

### Eample 1

### Encapsulation of Choline Bitartrate

To encapsulate choline bitartrate, a molten lipid mixture containing 95% hydrogenated palm oil and 5% distilled monoglyceride was sprayed onto choline bitartrate. The spraying continued until a desired amount of coating, depending upon the desired degree of protection of the encapsulate, is achieved. The choline bitartrate encapsulates were then allowed to cool. After cooling, the encapsulates were passed through a screen to break-up agglomerates.

The resulting encapsulates contain about 70% active choline bitartrate and about 30% lipid coating by weight of the encapsulate. The coating of the encapsulate stabilizes the choline and protects the choline when exposed to elevated temperatures, water, moisture, pressure, oxygen, and conditions encountered during preparation and storage of a dosage form.

### Example 2

### Release of Encapsulated Choline Bitartrate

The 5 hour release of the encapsulated choline bitartrate from Example 1 was determined. To determine the 5 hour release, the following procedure was used:

1) 1.0 gram of encapsulated choline bitartrate (to the nearest 0.01 mg) is placed into a 250 ml erlenmeyer flask. 100 ml of distilled water is added to the flask and the flask is sealed with a stopper.

2) The flask is then placed on a shaker for 5 hour with moderate shaking intensity.

3) At the end of 5 hours, the contents of the flask are filtered through a premoistened glass wool in a powder funnel. The elute is collected into a second flask. The first flask is then rinsed with 10 ml of water, and this is added to the second flask through the powder funnel/glass wool.

4) The solution in the second flask is then titrated with 0.1N NaOH. The end-point of the titration is determined by measuring the first inflexion in pH values, at approximately neutral pH.

The percent release can be calculate using the following equation:

$$Percent release = \frac{( mls of 0.1 N NaOH ) ( 0.1 ) ( 253 ) ( 100 )}{( mg of encapsulate ) \frac{( \% Choline Bitratrate )}{100}}$$

The 5 hour release of the encapsulated choline from Example 1 was determined to be less than 1%. Therefore, the nature of the coating offered significant protection to the choline when the encapsulated was exposed to water for 5 hours.

### Example 3

### Stability of a Tablet Containing Encapsulates of Choline Bitartrate

Multi-vitamin tablets containing heavy metals were prepared with different choline bitartrate encapsulates. Each tablet contained 55 mg of choline cation. Three choline bitartrate encapsulates were tested: a choline bitratrate encapsulate with a five hour release of 60%, a choline bitartrate encapsulate with a five hour release of 20%, and a choline bitartrate encapsulate with a five hour release of 1%, all of which were prepared according to Example 1, with varying levels of activity.

The tablets were stored at accelerated conditions (40° C. and 75% relative humidity) for 3 months. After 3 months, the quality of the tablets were evaluated.

The tablets containing encapsulated choline bitartrate with a five hour release of 60% exhibited significant discoloration and odor resembling amines. For the tablet prepared with choline bitartrate encapsulates having a five hour release of 20%, the tablet possessed less discoloration and odor, marginally failing tablet quality based upon discoloration and odor.

In contrast, the tablets containing encapsulated choline bitartrate with a five hour release less than 20%, in this case 1%, exhibited no signs of discoloration and odor development after 3 months storage at accelerated conditions. Therefore, choline bitartrate encapsulates with a five hour release of less than 20% are stable against conditions encountered during manufacturing and storage of a multi-vitamin tablet.

### Example 4

### Stability of a Tablet Containing Encapsulated Choline Bitartrate Versus Encapsulated Choline Chloride

To determine the stability of tablets containing choline bitartrate, tablets containing 70% choline bitartrate encapsulates were compared to tablets containing 40% choline chloride encapsulates. Each tablet contained 55 mg of choline cation. The lipid coating of both encapsulates contained a mixture of 95% hydrogenated palm oil and 5% distilled monoglyceride. Both encapsulates had similar levels of protection as determined by the 5 hour release test method.

The tablets containing either encapsulated choline bitartrate or choline chloride were stored at accelerated conditions (40° C. and 75% relative humidity) for 3 months. After 3 months, the quality of the tablets were evaluated.

The tablets containing the encapsulated choline chloride exhibited discoloration and odor resembling amines. Therefore, the choline chloride is not stabilized against conditions encountered during manufacturing and storage of the tablets.

In contrast, the tablets containing encapsulated choline bitartrate exhibited no signs of discoloration and odor development after 3 months storage at accelerated conditions. Therefore, at the same level of encapsulate protection, the low hygroscopic choline salt, choline bitartrate, was significantly more stable against conditions encountered during manufacturing and storage of the tablet.

Thus, while there have been described what are presently believed to be the preferred embodiments of the invention, changes and modifications can be made to the invention and other and further embodiments will be known to those skilled in the art, which fall within the spirit of the invention and it is intended to include all such other changes and modifications and embodiments as come within the scope of the claims as set forth herein below.

What is claimed:

1. A method of preparing a dosage form comprising choline, wherein said choline is stabilized against conditions encountered during manufacturing and storage of said dosage form, the method comprising:
a) encapsulating a low hygroscopic choline salt with a coating comprising a lipid of a character and in an amount to provide a stabilized encapsulated low hygroscopic choline salt having a release of less than about 20% when said encapsulate is exposed to water for five hours; and
b) combining a plurality of said encapsulated low hygroscopic choline salt with other ingredients,
whereby said dosage form comprising choline is prepared.

2. A method according to claim 1, wherein said choline is stabilized for at least about three months when said dosage form is stored under accelerated conditions.

3. A method according to claim 1, wherein said low hygroscopic choline salt is choline bitartrate.

4. A method according to claim 1, wherein said lipid comprises vegetable oil.

5. A method according to claim 4, wherein said vegetable oil is hydrogenated palm oil.

6. A method according to claim 1, wherein said coating further comprises additives.

7. A method according to claim 1, wherein said release is less than about 15%.

8. A method according to claim 1, wherein said release is less than about 10%.

9. A method according to claim 1, wherein said release is less than about 5%.

10. A method according to claim 1, wherein said release is less than about 1%.

11. A method according to claim 1, wherein said dosage form is a tablet.

12. A method according to claim 11, wherein said tablet is a multi-vitamin and mineral supplement.

13. A method according to claim 1, wherein said dosage form is a gel capsule

14. A method according to claim 13, wherein said gel capsule is a multi-vitamin and mineral supplement.

15. A method according to claim 1, wherein said dosage form comprises at least one heavy metal.

16. A method according to claim 15, wherein said heavy metal is selected from the group consisting of copper, iron, magnesium, manganese, molybdenum, zinc, nickel, selenium, chromium, tin, vanadium, and combinations thereof.

17. A method according to claim 1, wherein said dosage form comprises at least about 5 mg of choline cation.

18. A method according to claim 1, wherein said dosage form comprises at least about 20 mg of choline cation.

19. A method according to claim 1, wherein said dosage form comprises at least about 55 mg of choline cation.

20. A method according to claim 1, wherein said dosage form comprises at least about 110 mg of choline cation.

21. A dosage form comprising choline, wherein said choline is stabilized against conditions encountered during manufacturing and storage of said dosage form, by a method comprising:
a) encapsulating a low hygroscopic choline salt with a coating comprising a lipid of a character and in an amount to provide a stabilized encapsulated low hygroscopic choline salt having a release of less than about 20% when said encapsulate is exposed to water for five hours; and
b) combining a plurality of said encapsulated low hygroscopic choline salt with other ingredients.

22. A dosage form according to claim 21, wherein said choline is stabilized for at least about three months when said dosage form is stored under accelerated conditions.

23. A dosage form according to claim 21, wherein said low hygroscopic choline salt is choline bitartrate.

24. A dosage form according to claim 21, wherein said lipid comprises vegetable oil.

25. A dosage form according to claim 24, wherein said vegetable oil is hydrogenated palm oil.

26. A dosage form according to claim 21, wherein said coating further comprises additives.

27. A dosage form according to claim 21, wherein said release is less than about 15%.

28. A dosage form according to claim 21, wherein said release is less than about 10%.

29. A dosage form according to claim 21, wherein said release is less than about 5%.

30. A dosage form according to claim 21, wherein said release is less than about 1%.

31. A dosage form according to claim 21, wherein said dosage form is a tablet.

32. A dosage form according to claim 31, wherein said tablet is a multi-vitamin and mineral supplement.

33. A dosage form according to claim 21, wherein said dosage form in a gel capsule.

34. A dosage form according to claim 33, wherein said gel capsule is a multi-vitamin and mineral supplement.

35. A dosage form according to claim 21, wherein said dosage form comprises at least one heavy metal.

36. A dosage form according to claim 35, wherein said heavy metal is selected from the group consisting of copper, iron, magnesium, manganese, molybdenum, zinc, nickel, selenium, chromium, tin, vanadium, and combinations thereof.

37. A dosage form according to claim 21, wherein said dosage form comprises at least about 5 mg of choline cation.

38. A dosage form according to claim 21, wherein said dosage form comprises at least about 20 mg of choline cation.

39. A dosage form according to claim 21, wherein said dosage form comprises at least about 55 mg of choline cation.

40. A dosage form according to claim 21, wherein said dosage form comprises at least about 110 mg of choline cation.
